(19) United States
(12) Patent Application Publication
(10) Pub. No.: US 2019/0365689 A1
(43) Pub. Date: Dec. 5, 2019

(54) USE OF APE/REF-1 REDOX SPECIFIC INHIBITORS FOR TREATING METASTATIC PROSTATE CANCER

(71) Applicant: Indiana University Research and Technology Corporation, Indianpolis, IN (US)

(72) Inventors: Mark R. Kelley, III, Zionsville, IN (US); Travis Jerde, Carmel, IN (US); Melissa L. Fishel, Fishers, IN (US)

(21) Appl. No.: 16/479,987

(22) PCT Filed: Jan. 25, 2018
(86) PCT No.: PCT/US2018/015205

§ 371 Date: Jul. 23, 2019

Related U.S. Application Data
(60) Provisional application No. 62/450,125, filed on Jan. 25, 2017.

(51) Int. Cl. A61K 31/20; A61K 31/197; A61P 35/04

(52) U.S. Cl. CPC A61K 31/20; A61P 35/04; A61K 31/197

(57) ABSTRACT

Methods of using redox APE1/Ref-1 inhibitors to treat prostate cancer, and particularly, metastatic prostate cancer, are disclosed. Particularly, small molecule inhibitors of APE1/Ref-1 redox activity have been found to decrease cell proliferation and induce cell cycle arrest in metastatic prostate cancer cell lines. Further, these APE1/Ref-1 redox inhibitors can be used to reduce expression of survivin, which has been shown to be overexpressed in primary and metastatic tumors.

### CROSS REFERENCE TO RELATED APPLICATIONS

This application claims the benefit to U.S. Provisional Patent Application No. 62/450,125, filed on Jan. 25, 2017, which is hereby incorporated by reference in its entirety.

### STATEMENT OF GOVERNMENT SUPPORT

This invention was made with government support under W81XWH-14-1-0525 awarded by the U.S. ARMY Medical Research & Material Command (MRMC). The government has certain rights in the invention.

### BACKGROUND OF THE DISCLOSURE

The present disclosure relates generally to methods of using redox APE1/Ref-1 inhibitors to treat prostate cancer, and particularly, metastatic prostate cancer. Particularly, small molecule inhibitors of APE1/Ref-1 redox activity, APX3330 and APX2009, have been found to decrease cell proliferation and induce cell cycle arrest in metastatic prostate cancer cell lines. Further, the present disclosure relates to methods of using these APE1/Ref-1 redox inhibitors to reduce expression of survivin, which has been shown to be overexpressed in primary and metastatic tumors.

Prostate cancer (PCa) is the most common male malignancy and the second leading cause of cancer-related death of men in the western hemisphere. Small prostatic carcinomas exist in up to 29% of men in their thirties and 64% of men in their sixties with most of these carcinomas being indolent and curable by surgery or radiation. However, some men develop an aggressive phenotype that metastasizes and becomes incurable once colonizing the bone. In rare instances, prostate cancers can metastasize to the brain and in even rare cases, liver, lung, and kidney. In general, any spread of prostate cancers outside the prostate bed can be referred to as “metastatic prostate cancer”). In particular, bone metastases produce osteoblastic lesions that are associated with high morbidity and high mortality and attempts at delaying this tumor progression with chemotherapeutic agents have only prolonged survival for a few months. This necessitates a better understanding of the disease in order to create effective treatments for the aggressive phenotype where conventional therapeutics have failed.

Recently, it has been shown that reduction-oxidation (redox) regulation of critical transcriptional activators plays an essential role in cell proliferation and survival in a number of different cancers, including prostate cancer. Apurinic/apyrimidinic endonuclease 1 redox factor 1 (APE1/Ref-1) is a multifunctional protein that participates in DNA repair and redox transcriptional regulation. APE1/Ref-1 has been implicated in the development and progression of numerous cancer types along with being conversely correlated to tumor radiation and chemotherapy sensitivity and is known to be overexpressed in prostate cancer. APE1/Ref-1 redox regulation of cysteine residues within the DNA binding domain or transactivation domain is essential for full transcriptional activation of certain transcriptional activators including the oncogenic transcriptional activators AP-1, HIF-1α, NFκB and STAT3.

Additional treatments include androgen deprivation therapies and microtubule-targeting agents, which prolong survival of the subject, but resistance to these therapeutics is inevitable. It is thought that this resistance is driven by aberrant survival signaling and the induction of survival proteins in the cancer cells, which allows for the cancer cells to evade cell death and is crucial for tumor progression.

Survivin, an Inhibitor of Apoptosis (IAP) family member, is overexpressed in prostate cancer and has been implicated in resistance to various chemotherapeutic and pro-apoptotic agents. Survivin is classically known as an inhibitor of caspases due to its single BIR domain, but recently survivin has been found to be crucial in cell cycle progression as a member of the chromosomal passenger complex.

Based on the foregoing, it would be beneficial for a treatment for cancer, and particularly, metastatic prostate cancer, that not only decreases cancer cell proliferation, but also downregulates the expression of survivin such to limit the cancer cell's ability to develop resistance to the treatment. It would be further advantageous if the treatment could decrease transcriptional activity of oncogenic transcriptional activators such as NF-κ3 and STAT3.

### BRIEF DESCRIPTION OF THE DISCLOSURE

The present disclosure is generally related to the use of small molecule inhibitors of APE1/Ref-1 redox activity to decrease cancer cell proliferation and induce cell cycle arrest in metastatic prostate cancer cell lines. Further, the small molecule inhibitors can be administered to decrease transcriptional activity of oncogenic transcriptional activators and downregulate survivin expression. These effects lead to sensitizing drug-resistant prostate cancer to chemotherapy, and as such, the use of these small molecule inhibitors can be used in combination with known therapeutic agents for treating prostate cancer.

Accordingly, in one aspect, the present disclosure is directed to a method of treating metastatic prostate cancer in a subject in need thereof. The method comprises administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In another aspect, the present disclosure is directed to a method of decreasing cancer cell proliferation in a subject in need thereof. The method comprises administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In another aspect, the present disclosure is directed to a method of reducing survivin expression in a subject in need thereof. The method comprises administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In another aspect, the present disclosure is directed to a method of decreasing NFκB expression in a subject having metastatic prostate cancer. The method comprises administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In yet another aspect, the present disclosure is directed to a method of decreasing STAT3 expression in a subject having metastatic prostate cancer, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIGS. 1A & 1B show that APE1/Ref-1 and survivin were nuclear and cytoplasmic localized in human prostate cancer. FIG. 1A: (1,2) Hematoxylin and Eosin staining represented non-diseased (peripheral zone taken from cystoprostatectomy) and cancerous human prostate specimens. Scale bar=10 μM. Immunofluorescent images of stained non-diseased and cancerous sections (1′-3′) for APE1/Ref-1 and survivin. Scale bar=25 μm, n=12. FIG. 1B: Cellular fractionation representing basal survivin and APE1/Ref-1 protein localization in cancerous (PC-3, C4-2 and LNCaP) and non-cancerous (E7) prostatic cell lines. MEK 1/2 (cytoplasmic), Lamin B1 (nuclear) and Histone H3 (chromatin bound) were used as controls for each subcellular fraction.

FIG. 1C shows that APE1/Ref-1 and survivin were overexpressed in prostate cancer cells Immunoblot example of basal survivin and APE1/Ref-1 protein levels between the prostatic cell lines. Representative of three determinations with densitometry quantification. N=3, *-denoting p<0.05 (PC-3, C4-2 and LNCaP vs. E7) as assessed by ANOVA.

FIG. 2A depicts a Methylene blue assay. PC-3 and C4-2 cells were seeded 1,000-20,000 per well. Media was then removed and cells were fixed with methanol for 10 minutes and stained with 100 μL of 0.05% of methylene blue (LC16920-1 diluted in 1×PBS) for 1 hour. The cells were then washed three times with water and allowed to air dry overnight. Representative pictures were taken. 100 μL of 0.5N HCl was added to each well to dissolve the methylene blue stain and absorbance (@630 nm) was measured via spectrophotometry. Equations were derived from these trend lines and used to calculate relative cell numbers in subsequent experiments.

FIGS. 2B-2E show that APE1/Ref-1 redox function specific inhibitors decreased cell number in a concentration dependent manner PC-3 (FIG. 2B), C4-2 (FIG. 2C), LNCaP (FIG. 2D) and E7 (FIG. 2E) cell lines were treated with increasing concentrations of redox-specific inhibitor APX3330, its more potent analogue APX2009, and inactive analogue RN7-58 for 5 days (N=3). The cells were fixed and stained with methylene blue and measured via spectrophotometry. IC₂₅ and IC₅₀ were determined as the concentrations of drug at which there was a 25% and 50% reduction in absorbance compared to DMSO and were used for subsequent experiments. n=3. EC50s were compared between the drugs: * denotes p<0.05 drug EC50 versus RN7-58, while ¶ denotes p<0.05, APX3330 versus APX2009.

FIGS. 3A-3D show that treatment with APX3330 and APX2009 decreased survivin protein levels. PC-3 (FIG. 3A), C4-2 (FIG. 3B), LNCaP (FIG. 3C) and E7 (FIG. 3D) cell lines were treated with DMSO, or the growth inhibitory IC₂₅ and IC₅₀ drug concentrations of APX3330 or APX2009 for 48 hours Immunoblotting for survivin, APE1/Ref-1 and Actin as labeled. Representative of three determinations with densitometry quantification, N=3, *-denoting p<0.05 (DMSO vs. IC₂₅ and IC₅₀ Drug Concentrations) within ANOVA.

FIGS. 4A-4C show that APE1/Ref-1 siRNA knockdown decreased cell proliferation and surviving protein levels. FIG. 4A: Separate aliquots of PC-3 and C4-2 cell lines were transfected with two distinct sequences of 50 nM APE1/Ref-1 siRNA (verified >70% knockdown by immunoblotting) and growth was compared to scrambled siRNA-transfected cells. n=3, *-denoting p<0.05 within ANOVA (Scr vs siAPE#1), #-denoting p<0.05 within ANOVA (Scr vs siAPE#2). FIG. 4B: Representative pictures of fixed and methylene blue stained C4-2/PC-3 scrambled siRNA (Scr), survivin siRNA #1 (siAPE1 #1) and #2 (siAPE1 #2). FIG. 4C: Immunoblotting was performed using antibodies for APE1/Ref-1, survivin and GAPDH as labeled after 72 hours post-transfection.

FIGS. 5A-5C show that APE1/Ref-1 redox inhibition induced G1 cell arrest. FIG. 5A: PC-3 and C4-2 cell lines were treated with DMSO or APX2009 (9 and 14 μM, respectively) for 48 hours. Representative images were taken at 20× Magnification. Scale bar=50 μm. FIG. 5B: Immunoblotting was performed and membranes were probed with antibodies for Cleaved Caspase 3, Total Caspase, Cyclin B1, Cdc2, survivin and Actin as labeled. FIG. 5C: PC-3 and C4-2 cells were treated with DMSO or APX2009 (9 and 14 μM, respectively) for 48 hours and then collected and stained with RNAse/PI wash. Flow Cytometry was then performed. N=3, *-denoting p<0.05 by unpaired Student's t-Test.

FIGS. 6A-6E show that APE1/Ref-1 redox inhibition decreased survivin protein levels via NFκB. FIG. 6A: C4-2 cell line was treated with DMSO or APX2009 (14 μM) for 12 hours. RNA was isolated and RT-PCR for survivin was performed with HPRT1 as the reference gene. n=6, *-denoting p<0.05 by unpaired Student's t-test. FIG. 6B: Immunoblot validation of APE1/Ref-1 and p65 Co-Immunoprecipitation (Co-IP) reactions. A 5% sample of the total input of each reaction (Input) and the total IP reaction (IP) were loaded for each reaction. Beads lacking a conjugated antibody (Mock) and generic IgG (IgG) were used as negative controls for each IP experimental reaction, APE1 antibody (top blots) and p65 (bottom blots). FIG. 6C: C4-2 cell line was treated with DMSO, APX2009 (8 and 14 μM) or PDTC (50 and 100 μM) for 72 hours and cells were fixed and methylene blue was performed. n=3, *-denoting p<0.05 (DMSO vs 8 and 14 μM APX2009) and #-denoting p<0.05 (DMSO vs. 50 and 100 μM PDTC) as assessed by ANOVA. FIG. 6D: C4-2 cells were transfected with NFκB-Luc construct and co-transfected with a Renilla vector, pRL-TK. After 16 hours, cells were treated with growth inhibitor IC₅₀ concentrations of APX2009 and PDTC for 24 hours, and Firefly and Renilla luciferase activities were assayed using Renilla luciferase activity for normalization. All transfection experiments were performed in triplicate and repeated 3 times in independent experiments. Data are expressed as Relative Luciferase Units (RLU) normalized to DMSO showing the mean±SEM. n=3, *-denoting p<0.05 (DMSO vs. 14 μM APX2009) and #-denoting p<0.05 (DMSO vs. 100 μM PDTC) within unpaired Students t-test. FIG. 6E: C4-2 cell line was treated with APX2009 (14 μM) and NFκB-selective inhibitor PDTC (100 μM) for 48 hours Immunoblotting was performed with antibodies for survivin, p65, APE1/Ref-1 and Actin as labeled. Data presented are representative of three determinations with densitometry quantification, n=33, *-denoting p<0.05 (DMSO vs. 14 μM APX2009) and #-denoting p<0.05 (DMSO vs. 100 μM) as assessed by unpaired Student's t-test.

FIG. 7 depicts cellular localization of NFB is altered upon APE1/Ref-1 redox inhibition. C4-2 cells were treated with either DMSO or 14 M APX2009 for 48 hours and then fixed for immunofluorescence (p65=Green and APE1/Ref-1=Red). P65 and APE1/Ref-1 were found to be co-localized in the nucleus. However, upon treatment with APX2009, p65 nuclear localization was diminished.

FIGS. 8A-8C show that in vivo treatment with APX2009 reduced survivin protein levels and BrdU incorporation in C4-2 xenograft tumors. C4-2 xenograft tumors were treated with vehicle (Propylene Glycol Kolliphor HS15 Tween 80 (PKT)) or APX2009 (25 mg/kg, IP bid) for 5 days (n=3). Tumors were removed and processed for either immunofluorescence or immunoblotting. FIG. 8A: APE1/Ref-1 and survivin protein levels were measured using immunoblotting as labeled (Left). Data was presented graphically (Right), *-denoting p<0.05 by unpaired Student's t-Test. FIG. 8B: Hematoxylin and Eosin staining and immunofluorescence were performed using APE1/Ref-1 (red) and survivin (green) specific antibodies on vehicle and APX2009 groups. Representative images were taken. White arrows are depicting survivin nuclear staining patterns. Scale bar H&E=10 μM. Scale bar immunofluorescence=25 μm. FIG. 8C: Mice were injected with BrdU 2 hours prior to sacrifice and tumors were collected and stained for BrdU incorporation (red). Scale bar=100 μm. ImageJ Nucleus Counter was used to quantify number of BrdU+ nuclei and total nuclei per image. n=3, *-denoting p<0.05 by unpaired Student's t-test.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

### A. Definitions

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “tissue” or “cell sample” refers to a collection of similar cells obtained from a tissue of a subject or patient. The source of the tissue or cell sample may be blood or any blood constituents (e.g., whole blood, plasma, serum) from the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

As used herein, the terms “control”, “control cohort”, “reference sample”, “reference cell”, “reference tissue”, “control sample”, “control cell”, and “control tissue” refer to a sample, cell or tissue obtained from a source that is known, or believed, to not be afflicted with the disease or condition for which a method or composition of the present disclosure is being used to identify and/or treat. The control can include one control or multiple controls. In one embodiment, a reference sample, reference cell, reference tissue, control sample, control cell, or control tissue is obtained from a healthy part of the body of the same subject or patient in whom a disease or condition is being identified/treated using a composition or method of the present disclosure. In one embodiment, a reference sample, reference cell, reference tissue, control sample, control cell, or control tissue is obtained from a healthy part of the body of an individual who is not the subject or patient in whom a disease or condition is being identified/treated using a composition or method of the invention.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, a companion animal, an experimental animal, etc. The subject can be suspected of having or at risk for having a condition (such as metastatic prostate cancer) or be diagnosed with a condition (such as metastatic prostate cancer). The subject can also be suspected of having or at risk for having metastatic prostate cancer. According to one embodiment, the subject to be treated according to this invention is a human.

The term “inhibit”, and derivatives thereof, includes its generally accepted meaning, which includes reducing, decreasing, prohibiting, preventing, restraining, and slowing, stopping, or reversing progression or severity. Thus, the present methods include both medical therapeutic and prophylactic administration, as appropriate. As such, a subject in need thereof, as it relates to the therapeutic uses herein, is one identified to require or desire medical intervention.

An “effective amount” is that amount of an agent necessary to inhibit and/or reduce the symptoms of the pathological diseases and disorders herein described (e.g., metastatic prostate cancer). When at least one additional therapeutic agent is administered to a subject, such agents may be administered sequentially, concurrently, or simultaneously, in order to obtain the benefits of the agents.

As used herein, “treating”, “treatment”, “alleviating”, “alleviate”, and “alleviation” refer to measures, wherein the object is to prevent or slow down (lessen) the targeted pathologic condition or disorder or relieve some of the symptoms of the disorder (e.g., metastatic prostate cancer). Those in need of treatment can include those already with the disorder as well as those prone to have the disorder, those at risk for having the disorder and those in whom the disorder is to be prevented.

Apurinic/apyrimidinic endonuclease 1 redox factor 1 (APE1/Ref-1) is a multifunctional protein that has recently been found to be essential in activating oncogenic transcription factors. Furthermore, the blockade of APE1/Ref-1's redox activity has been shown to reduce growth-promoting, inflammatory and anti-apoptotic activities in cells. The present disclosure generally relates to methods of targeting apurinic/apyrimidinic endonuclease1/redox effector factor 1 (APE1/Ref-1). More particularly, by inhibiting APE1/Ref-1, it is believed that prostate cancer cell growth and survival can be inhibited.

Accordingly, in one embodiment, the present disclosure is directed to a method of treating metastatic prostate cancer in a subject in need thereof. The method includes administering to the subject an effective amount of an apurinic/apyrimidinic endonuclease 1 redox factor 1 (APE/Ref-1) inhibitor, pharmaceutically acceptable salts or pharmaceutically acceptable solvates thereof, which selectively inhibits the redox function of Ape1/Ref-1.

The redox function of APE1/Ref-1 was found to be selectively inhibited by 3-[(5-(2,3-dimethoxy-6-methyl1,4-benzoquinoyl)]-2-nonyl-2-proprionic acid, (hereinafter “E3330” or “3330” or “APX3330”) and/or [(2E)-2-[(3-methoxy-1,4-dioxo-1,4-dihydronaphthalen-2-yl)methylidene]-N,N-diethylpentanamide] (hereinafter “APX2009”). Further information on APX3330 may be found in Abe et al., U.S. Pat. No. 5,210,239, and information on APX2009 may be found in Kelley et al., J Pharmacol Exp Ther. 2016 November, 359(2): 300-309, each incorporated herein by reference to the extent they are consistent herewith.

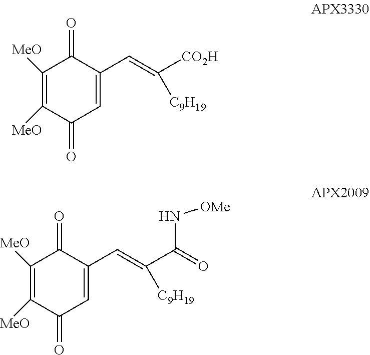

Interestingly, the Examples below indicate that selective blocking of the redox function of Ape1/Ref-1 does not cause any or any appreciable apoptosis in normal cells. One very well might expect that the selective blocking resulting in increased apoptosis in cancerous cells would also impair normal cells. However, this was found not to be the case.

Further, a key feature of prostate cancer progression is the induction and activation of survival proteins, most prominently the inhibitor of apoptosis family member survivin. Survivin is known to be differentially regulated in various tissues and in response to external stimuli. It has been shown in the literature that survivin can be transcriptionally regulated by a number of transcription factors including Sp-1, STAT3 and NFκB. In the present disclosure, evidence is provided that survivin is being transcriptionally regulated by NFκB. It is further recognized that other transcription factors may also be playing a role. As shown in the Example below, survivin mRNA is significantly reduced, p65 cellular localization is disrupted and NFκB luciferase activity is decreased after treatment with the APE1/Ref-1 inhibitors, APX3330 and APX2009.

Accordingly, in one embodiment, the present disclosure is generally directed to a method of reducing survivin expression in a subject in need thereof. The method includes administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In another embodiment, the present disclosure is generally directed to a method of decreasing NFκB expression in a subject having metastatic prostate cancer. The method includes administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

In yet another embodiment, the present disclosure is generally directed to a method of decreasing STAT3 expression in a subject having metastatic prostate cancer. The method includes administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

Additionally, metastatic prostate cancer could have varying responses to drug treatment relative to localized disease. Prostate cancer metastasizes primarily to lymph nodes and to bones. Because the behavior of prostate cancer cells is highly dependent upon signals from the environment that they are living in, and the bone and lymph nodes have very unique tissue environments that are clearly distinct from the prostatic environment from which prostate cancer cells originated, it may be possible that prostate cancer cells could have completely different responses to a given drug in the bone environment than in the prostate environment.

Where subject applications are contemplated, particularly in humans, it will be necessary to prepare pharmaceutical compositions in a form appropriate for the intended application. Generally, this will entail preparing compositions that are essentially free of impurities that could be harmful to a subject.

The inhibitors (i.e., APX3330 & APX2009) can be administered in the methods of the present disclosure orally, intravenously, intramuscularly, intrapleurally or intraperitoneally at doses based on the body weight and degree of disease progression of the subject, and may be given in one, two or even four daily administrations. For example, in some embodiments, the inhibitor is APX3330 and is administered in amounts ranging from about 5 μM to about 100 μM, including about 25 mg/kg. In other embodiments, the inhibitor is APX2009 and is administered in amounts ranging from about 1 μM to about 30 μM, including from about 9 μM to about 14 μM.

One will generally desire to employ appropriate salts and buffers to render agents stable and allow for uptake by target cells. Aqueous compositions of the present disclosure comprise an effective amount of the agent, dissolved or dispersed in a pharmaceutically acceptable carrier or aqueous medium. Such compositions also are referred to as innocuously. The phrase pharmaceutically or pharmacologically acceptable refers to molecular entities and compositions that do not produce adverse, allergic, or other untoward reactions when administered to a subject. As used herein, pharmaceutically acceptable carrier includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Compositions for use in the present disclosure may include classic pharmaceutical preparations. Administration of these compositions according to the present disclosure will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, as described herein.

For example, the inhibitors can be formulated with common excipients, diluents, or carriers, and formed into tablets, capsules, suspensions, powders, and the like. Examples of excipients, diluents, and carriers that are suitable for such formulations include the following: fillers and extenders such as starch, sugars, mannitol, and silicic derivatives; binding agents such as carboxymethyl cellulose and other cellulose derivatives, alginates, gelatin, and polyvinyl pyrrolidone; moisturizing agents such as glycerol; disintegrating agents such as calcium carbonate and sodium bicarbonate; agents for retarding dissolution such as paraffin; resorption accelerators such as quaternary ammonium compounds; surface active agents such as cetyl alcohol, glycerol monostearate; adsorptive carriers such as kaolin and bentonite; and lubricants such as talc, calcium and magnesium stearate, and solid polyethyl glycols.

The inhibitors may also be administered parenterally or intraperitoneally. Solutions of the inhibitors as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In some particularly suitable embodiments, the form is sterile and is fluid to the extent that easy administration via syringe exists. It can be stable under the conditions of manufacture and storage and can be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the inhibitors in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The oral administration of the inhibitors may include incorporating the inhibitors with excipients and used in the form of non-ingestible mouthwashes and dentifrices. A mouthwash may be prepared incorporating the inhibitors in the required amount in an appropriate solvent, such as a sodium borate solution (Dobell's Solution). Alternatively, the inhibitors may be incorporated into an antiseptic wash containing sodium borate, glycerin and potassium bicarbonate. The inhibitors may also be dispersed in dentifrices, including gels, pastes, powders and slurries. The inhibitors may be added in a therapeutically effective amount to a paste dentifrice that may include water, binders, abrasives, flavoring agents, foaming agents, and humectants.

The compositions for use in the present disclosure may be formulated in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amounts as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media, which can be employed, will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, general safety and purity standards as required by FDA and foreign counterpart agencies.

In some aspects, as noted above, the APE1/Ref-1 inhibitor is administered in combination with one or more additional therapeutic agents. Particularly, it has been found that the APE1/Ref-1 inhibitor decreases survivin protein levels in the cancer cells, cancer cells are sensitized to chemotherapy. Accordingly, in some embodiments, the APE1/Ref-1 inhibitor can be combined with one or more chemotherapeutic agents (e.g., cyclophosphamide, dexamethasone, vincristine, doxorubicin, methotrexate, platinum-based compounds (e.g., cisplatin, carboplatin), doxetaxel (and other taxel related drugs (e.g., carazitaxel, taxotere), steroids (e.g., prednisone), antiandrogens, anti-LHRH, ionizing radiation, radiation drugs (e.g., Xofigo, metastron and quadramet) provenge (sipuleucel-T), and combinations thereof).

As noted above, the APE/Ref-1 inhibitor can further be used to reduce STAT3 expression levels. Accordingly, in some aspects, the APE1/Ref-1 inhibitor can be administered in combination with additional therapeutic agents to further reduce STAT3 expression. Exemplary additional therapeutic agents include an inhibitor of signal transducer and activator of transcription 3 (STAT3) (e.g., 6-(4-amino-4-methyl-1-piperidinyl)-3-(2,3-dichlorophenyl)-2-pyrazinamine (SHP099); 2-Hydroxy-4-(((4-methylphenyl)sulfonyloxy)acetyl)amino)-benzoic acid/S3I-201, 6-Nitrobenzo[b]thiophene-1,1-dioxide/stattic, OCHROMYCINONE, 4-(N-(4-Cyclohexylbenzyl)-2-(2,3,4,5,6-pentafluoro-N-methylphenylsulfonamido)acetamido)-2-hydroxybenzoic acid; napabucasin).

### Example

In this Example, the effects of APE1/Ref-1 inhibitors, APX3330 and APX2009, on prostate cancer cell proliferation, survivin protein levels and NFκB activity were analyzed.

### Materials and Methods

Cell Lines

PC-3, LNCaP and C4-2 prostate cancer cell lines were purchased from and authenticated by the ATCC (Manassas, Va.). E7 prostate epithelial cells were received from Dr. David Jarrard, Department of Urology, University of Wisconsin-Madison. All cell lines were maintained at 37° C. in 5% CO₂ and grown in RPMI (Corning: Manassas, Va.) with 5% Fetal Bovine Serum (HyClone: Logan, Utah).

Drugs

APX3330, which is also called E3330, was synthesized as previously described in Luo et al., Antioxid Redow Signal. 2008; 10:11: 1853-1867. APX2009 was obtained from Apexian Pharmaceuticals LLC (Indianapolis, Ind.). Synthesis and description of APX2009 and RN7-58 has been described in Luo et al., Antioxid Redow Signal. 2008; 10:11: 1853-1867; Nyland et al., J Med Chem. 2010; 53:1200-1210. PDTC (Ammonium pyrrolidinedithiocarbamate) (ab141406) was obtained from Dr. Tao Lu (Indianapolis, Ind.) who purchased it from Abcam (Cambridge, Mass.).

Immunofluorescence

Human prostate specimens or C4-2 xenograft tumors were fixed in 10% formalin, processed routinely, embedded in paraffin, and serially sectioned at 5 μm via microtome. Tissues were subjected to heat-induced antigen retrieval in 10 mM citrate buffer (citrate buffer stock solution of monohydrate-free acid citric acid, sodium citrate dehydrate, pH 6.0) for 10 minutes followed by 10-minute rest. Sections were blocked at room temperature with a bovine serum albumin (BSA)-Donkey serum mixture for 2 hours and incubated with primary antibody overnight at 4° C. Primary antibodies and dilutions included rabbit survivin (1:100, Cell Signaling Technologies), mouse APE1/Ref-1 (1:200, Novus Biologicals), rabbit BrdU (1:200, Cell Signaling Technologies), and mouse PanCK (1:200, Cell Signaling Technologies). Sections were washed with 1×PBS (Phosphate-buffered saline)-TWEEN® and incubated with IgG Alexa 488 and IgG Alexa 594-conjugated secondary antibody against rabbit or mouse for 1 hour at room temperature (1:200, Invitrogen), followed by 10 minutes incubation with Hoechst 33258 nuclear stain (1 μg/ml). Tissues were washed with 1×PBS-TWEEN® and water and then covered with an aqueous medium/glass coverslip. The sections were analyzed by immunofluorescence.

Human Specimens

Human prostate specimens (n=12) were obtained with appropriate minimal risk institutional review board approval according to the approval and guidelines at Indiana University School of Medicine. Sections were cut from pre-existing paraffin-embedded human prostate tissues obtained as part of a prostatectomy or from prostate specimens removed collaterally from bladder cancer patients undergoing cystoprostatectomy as control human specimens. These controls were age-matched to the prostate cancer specimens and were verified by record to be naïve for pretreatment with Bacillus Calmette-Guérin (BCG) because these patients had presented first with muscle invasive bladder cancer. The controls were verified by pathology to be void of prostate cancer. The controls were verified by pathology to be void of prostate cancer, BPH, or prostatitis. All human specimens were stained with survivin and APE1/Ref-1 antibodies for immunofluorescence, as described above.

Immunoblotting

Prostate cells were homogenized in lysis buffer containing protease inhibitor (150 mM NaCl, 10 mM tris, 1 mM EDTA, 1 mM benzenesulfonyl fluoride, and 10 μg/ml each of aprotinin, bestatin, L-luecine, and pepstatin A) and 1% Triton X-100. Total protein concentration was determined by BCA (bicinchoninic acid) assay (Pierce, Rockford, Ill.). 10 μg/well of Protein were resolved by electrophoresis in 4-15% gradient polyacrylamide gels (Bio-Rad Laboratories). Proteins were transferred to polyvinylidene difluoride (PVDF) membranes, blocked for 24 hours [(10% Dry milk, 5% BSA, 0.05% NaN₃) in 1×PBS (2.7 mM KCl, 1.5 mM KH₂PO₄, 136 mM NaCl, 8 mM Na₂HPO₄)-TWEEN® 20] and incubated overnight with one of the following primary antibodies: mouse (3-actin (1:2500, ThermoFisher Scientific), mouse APE1/Ref-1 (1:1000, Novus Biologicals), rabbit survivin (1:500, Cell Signaling Technologies), rabbit Bcl-2 (1:500, Cell Signaling Technologies), rabbit Mcl-1 (1:500, Cell Signaling Technologies), rabbit Cleaved Caspase 3 (1:250, Cell Signaling Technologies), rabbit Total Caspase 3 (1:1000, Cell Signaling Technologies), rabbit Cyclin B1 (1:500, Cell Signaling Technologies), Cdc2 (1:1000, Cell Signaling Technologies) and rabbit GAPDH (1:1000, Cell Signaling Technologies). After blots were washed 6 times with PBS-TWEEN®, blots were incubated with donkey antibody against rabbit or mouse immunoglobulin G conjugated to horseradish peroxidase for 1 hour (1:10,000 dilution, Pierce) in nonfat dry milk, 1×PBS, and 0.05% TWEEN® 20. Peroxidase activity was detected via Pico chemiluminescence reagent (Pierce). Photo images were analyzed by densitometry.

Methylene Blue Assay (Cell Proliferation)

Prostate cells were seeded 1,000-5000 per well (cell line/experiment-dependent) and treated with one of APX3330, APX2009 or RN7-58 for 5 days. Media was then removed and cells were fixed with methanol for 10 minutes and stained with 100 μL of 0.05% of methylene blue (LC16920-1 diluted in 1×PBS) for 1 hour. The cells were then washed 3× with water and allowed to air dry overnight. 100 μL of 0.5 N HCl was added to each well to dissolve the methylene blue stain and absorbance (@630) was measured via spectrophotometry. The percent viabilities, normalized to DMSO, were graphed and IC₅₀ concentrations determined. DMSO control was not significantly different from media alone cells.

Reverse Transcription-PCR

RNA isolation was performed using RNeasy Mini Kit (Qiagen). 10 nanograms of total RNA was reverse transcribed using Superscript III One-Step RT-PCR System (ThermoFisher Scientific). Real-time PCR was performed using the TaqMan Gene Expression Assay (BIRC5 (Hs04194392_s1) and HPRT1 (Hs02800695_m1), ThermoFisher Scientific) and Applied Biosystems 7500 Fast Real-Time PCR System.

Co-Immunoprecipitation

Samples were co-immunoprecipitated using the Pierce Co-IP kit (Thermo Scientific). Additionally, the cells were washed twice with 1×PBS and the proteins were cross-linked using DTBP (Thermo Scientific, 5 mm, for 30 minutes on ice). DTBP was quenched by washing with cold inactivation buffer (100 mm Tris-HCl, pH 8, 150 mm NaCl) and 1×PBS. Cells were then lysed and the lysates added to columns and after extensive washing, the bound proteins were eluted and prepared for immunoblot analysis.

Luciferase Assay

C4-2 cells were co-transfected with constructs containing luciferase driven by NF-κB (pLuc-MCS with NF-κ3 responsive promoter; PathDetect cis-Reporting Systems, Stratagene, La Jolla, Ca) and a Renilla luciferase control reporter vector pRL-TK (Promega Corp., Madison, Wis.) at a 20:1 ratio by using Effectene Transfection Reagent (Qiagen; Valencia, Calif.). After 16 hours, cells were treated with increasing concentrations of APX2009 in serum free media for 24 hours. Firefly and Renilla luciferase activities were assessed by using the Dual Luciferase Reporter Assay System (Promega Corp.). Renilla luciferase activity was used for normalization and all transfection experiments were performed in triplicate and repeated 3 times in independent experiments.

Propidium Iodide Cell Cycle Analysis

PC-3 and C4-2 cells were treated with APX2009 (9 and 14 μM, respectively) for 48 hours. 500,000 cells were then aliquoted for cell cycle analysis and 0.1 mg/ml Propidium Iodide and 0.6% NP-40 PBS stain wash was added to the tubes. The cells were then centrifuged at 1900 rpms for 10 minutes with the brake on low and then decanted and blotted. RNAase and stain wash were added and cells incubated on ice for 30 minutes. Propidium Iodide intensity was measured via flow cytometry.

In Vivo Subcutaneous Tumor

2×10⁶ C4-2 cells were subcutaneously implanted in the hind flank of male athymic nude mice using a 100 μl volume of 50:50 solution of Matrigel: RPMI medium. When tumor volumes reached 150-200 mm³, the animals were treated every 25 mg/kg IP APX2009 or vehicle (Propylene Glycol Kolliphor HS15 Tween 80 (PKT)) every 12 hours for 5 days. BrdU was injected into the animals 2 hours prior to sacrifice and tumor tissues were analyzed for survivin levels (immunofluorescence and immunoblotting) and BrdU incorporation (immunofluorescence).

siRNA Transfection

All siRNA transfections were performed using the HiPerfect Transfection Reagent (Qiagen) protocol. Post-transfection C4-2 cells (1,000 per well) and PC-3 cells (1,500 per well) were replated in a 96-well plate and fixed daily up to 6 days and methylene blue assay was performed. Samples for immunoblotting were collected 72 hours post transfection of cancer cells with APE1/Ref-1 siRNA and scrambled siRNA control. Prevalidated APE1/Ref-1 siRNA (siAPE1 #2) was purchased from LifeTech (#s1446).

Statistics

Summary statistics are presented using the mean, median, and SD. Either a Student's t-test or ANOVA test was performed to compare the groups as appropriate. Statistical significance was assessed at the p<0.05.

### Results

APE1/Ref-1 and Survivin are Nuclear and Cytoplasmic Localized in Human Prostate Cancer

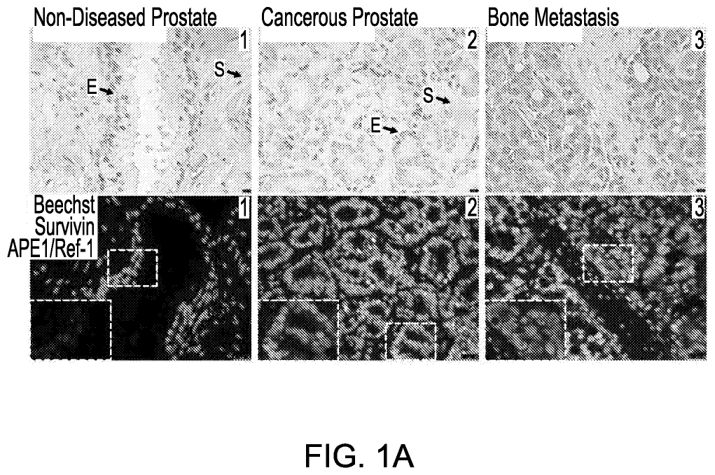

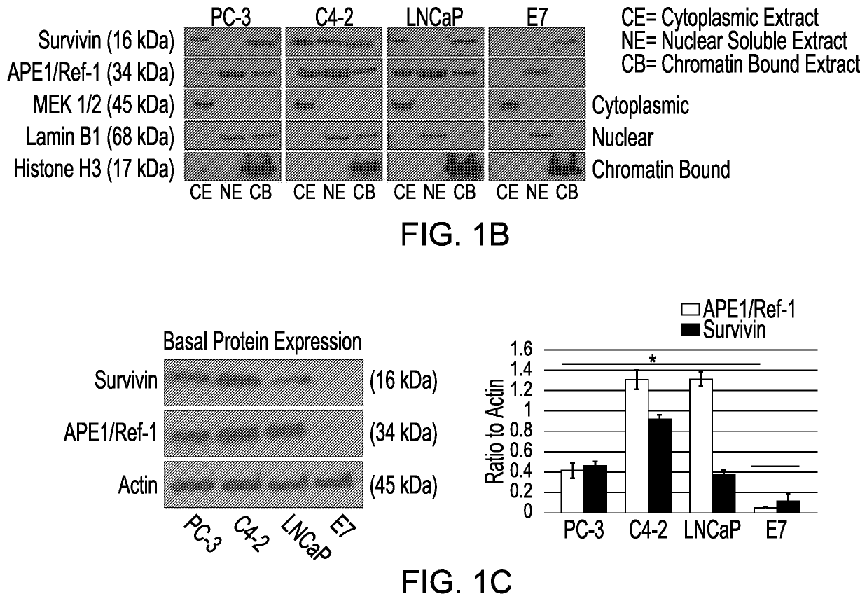

To confirm that APE1/Ref-1 and survivin protein expression is altered in prostate cancer, immunofluorescence was performed using human non-diseased and cancerous prostate specimens (FIG. 1A). It was found that APE1/Ref-1 is overexpressed in prostate cancer compared to non-diseased control prostates, and it co-localizes with survivin-expressing cells (71% co-localization in primary tumor specimens and over 99% in metastatic specimens). Expression of both proteins was primarily found to be nuclear and localized to in the epithelium, but in cancerous prostates cytoplasmic localization was observed (FIG. 1A, inlet). To determine if well-characterized prostatic cell lines displayed the same pattern, PC-3, C4-2, LNCaP and non-cancerous E7 cells were fractionated into cytoplasmic, nuclear soluble and chromatin bound fractions and immunoblotting was performed evaluating APE1/Ref-1 and survivin protein localization (FIG. 1B). E7 cell line is a normal prostatic epithelial cell line that was transformed using the human papillomavirus 16 (HPV16) E7 gene. MEK 1/2, Lamin B1 and Histone H3 were used as the respective controls for each fraction. APE1/Ref-1 protein localization was found to be in all three subcellular fractions in cancerous cell lines but only the nuclear soluble fraction in non-cancerous E7 cells. Survivin protein localization was primarily found in the cytoplasmic and chromatin bound fraction with some variable expression in the nuclear soluble fraction in the cancerous cell lines but localized only to the chromatin bound fraction in the non-cancerous E7 cells. This mirrors the expression pattern found in the human specimens. Additionally, APE1/Ref-1 and survivin protein levels were found to be significantly higher in PC-3, C4-2 and LNCaP cell lines compared to the E7 cell line (FIG. 1C).

APE1/Ref-1 Redox Inhibition Decreases Prostate Cancer Cell Proliferation

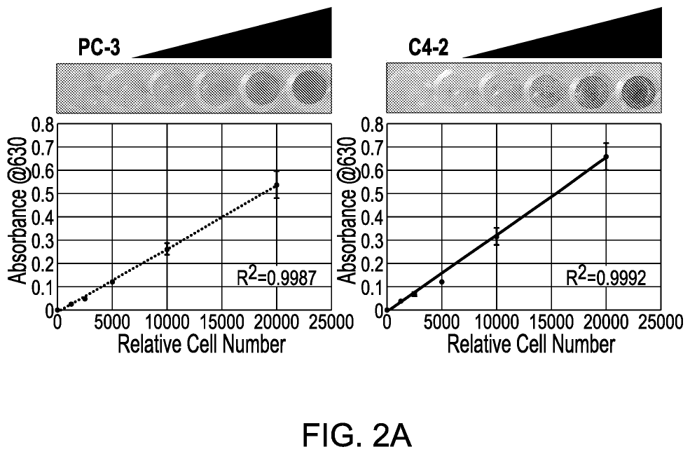

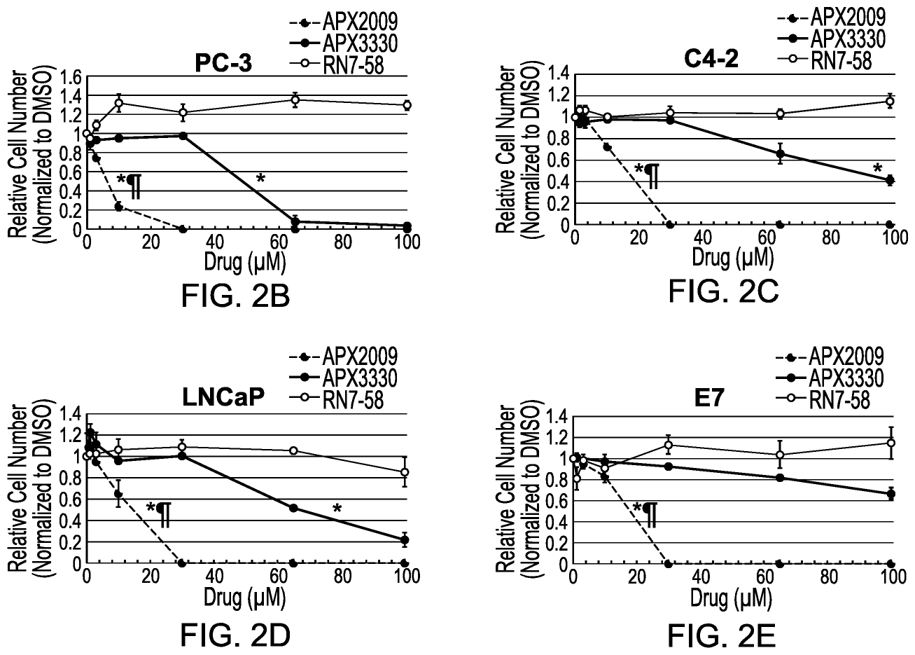

To determine if inhibition of APE1/Ref-1's redox function affects cell proliferation, prostatic cell lines were treated with increasing concentrations of redox-specific inhibitors, APX3330 and APX2009, for 5 days and cell number was measured via methylene blue assay (FIG. 2A). RN7-58, an inactive analogue of APX3330, was used as a negative control. It has been shown to have no effect on APE1/Ref-1 redox function. APX3330 and APX2009 inhibited cell proliferation in a concentration-dependent manner (FIGS. 2B-2E). Growth IC₂₅'s and IC₅₀'s were determined and arranged in Table 1. Student's t-test was performed to verify statistical IC₂₅ and IC₅₀ differences between APX3330 and APX2009. RN7-58 caused variable cell growth between cell lines but did not contain IC₂₅ or IC₅₀ drug concentrations. APX2009 was found to be 5-10 fold more efficacious than parent compound APX3330 in inhibiting cell proliferation. DMSO control was not significantly different from untreated cells.

TABLE 1

| | | APX3330 | APX2009 | P value |
|---|---|---|---|---|
| PC-3 | IC₂₅ | 36.0 +/− 1.0 | 2.2 +/− 0.5 | >0.0001 |
| | IC₅₀ | 54.7 +/− 1.6 | 8.9 +/− 0.7 | >0.0001 |
| C4-2 | IC₂₅ | 57.4 +/− 3.8 | 7.6 +/− 0.2 | 0.0002 |
| | IC₅₀ | 89.5 +/− 7.8 | 14.2 +/− 0.3 | 0.0006 |
| LNCaP | IC₂₅ | 43.8 +/− 4.2 | 6.3 +/− 1.6 | 0.0011 |
| | IC₅₀ | 71.9 +/− 7.2 | 13 +/− 1.2 | 0.0013 |
| E7 | IC₂₅ | 82.7 +/− 8.7 | 9.2 +/− 0.7 | 0.0011 |
| | IC₅₀ | >100 | 16.1 +/− 0.8 | — |

APE1/Ref-1 Redox-Specific Inhibitors Decrease Survivin Protein Levels

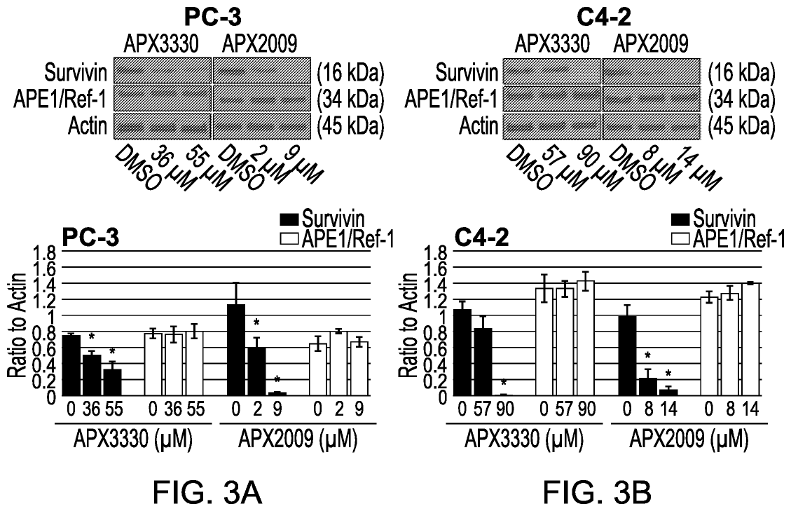

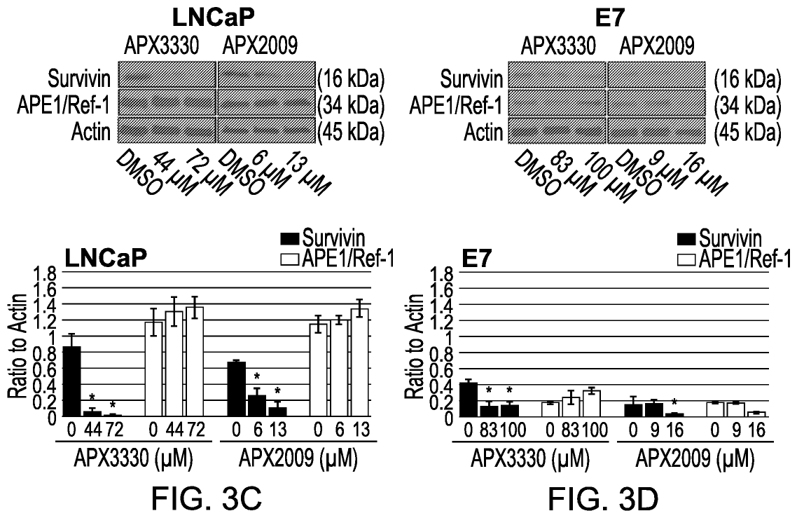

Prostate cancer cells treated with respective growth inhibitory IC₅₀ drug concentrations of APX3330 and APX2009 exhibited a significant decrease in survivin protein abundance within 48 hours compared to DMSO treated controls (FIGS. 3A-D). In contrast, prostate cancer cell total APE1/Ref-1 protein levels were not significantly altered with treatment.

APE1/Ref-1 siRNA Reduces Proliferation and Survivin Protein Levels

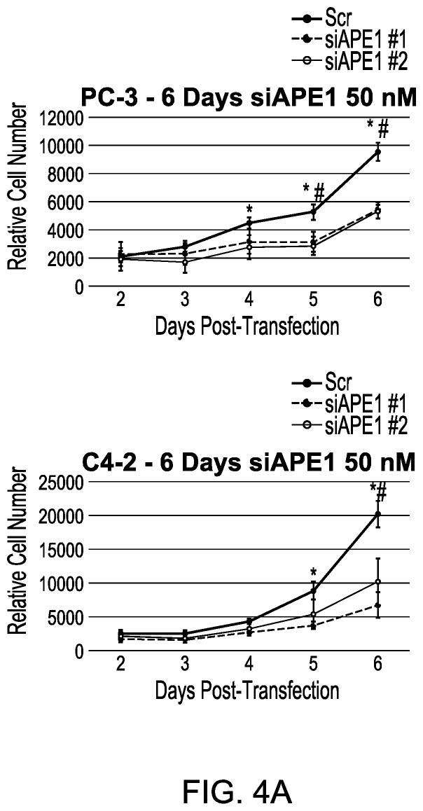

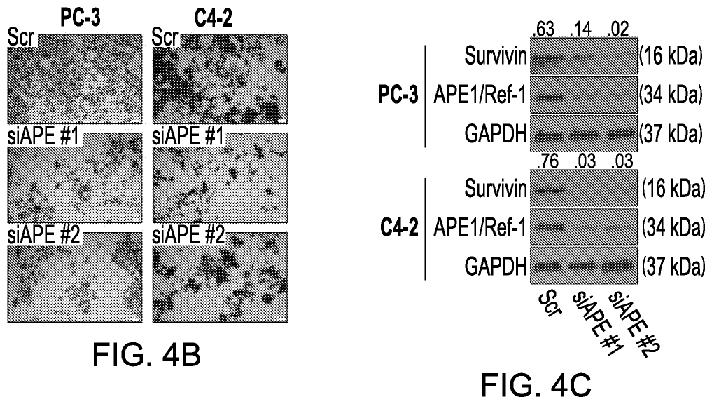

Using siRNA specific to APE1/Ref-1, it was analyzed if APE1/Ref-1 knockdown reduces cell growth and survivin protein levels. PC-3 and C4-2 cell lines were transfected with two distinct sequences of 50 nM APE1/Ref-1 siRNA (verified >70% knockdown by immunoblotting) and growth was compared to scrambled siRNA-transfected cells (FIG. 4A). Those cells transfected with APE1/Ref-1 siRNA grew at a significantly slower rate compared to those cells transfected with the scrambled siRNA. Representative pictures of fixed and methylene blue stained C4-2 and PC-3 scrambled siRNA (Scr), survivin siRNA #1 (siAPE1 #1) and #2 (siAPE1 #2) were taken (FIG. 4B) Immunoblotting was performed 72 hours post transfection and survivin protein levels were found to be decreased compared to scrambled control (FIG. 4C).

Treatment with APX2009 Induces G1 Cell Arrest, but not Cell Death

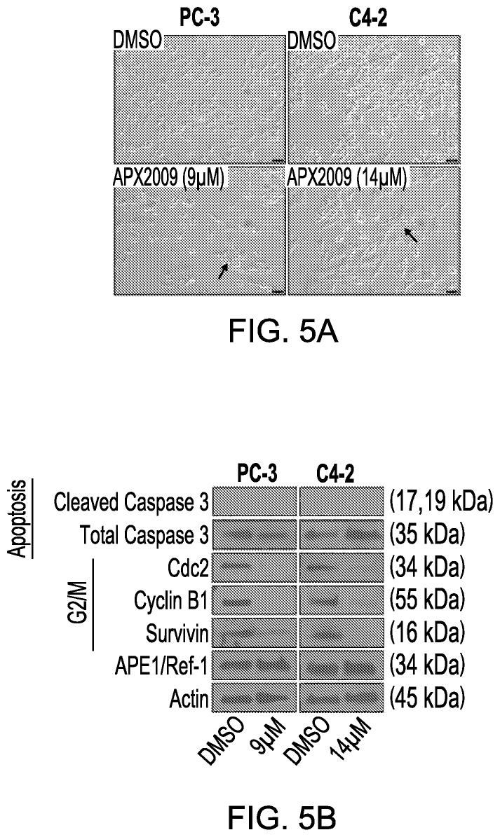

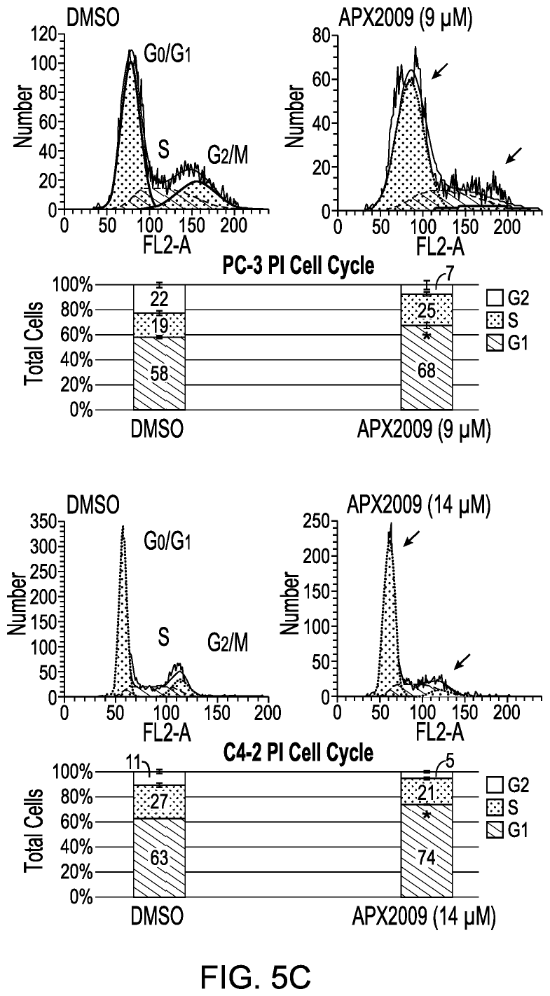

To determine if treatment with APX2009 resulted in cell death due to loss of survival signaling, PC-3 and C4-2 cells were treated with either DMSO or APX2009 (9 μM and 14 μM, respectively) for 48 hours (FIG. 5A) and cell lysates were collected for immunoblotting (FIG. 5B). After APX2009 treatment, both PC-3 and C4-2 cells displayed an altered, flattened cellular morphology. However, treatment with these compounds did not induce cell death as determined by both a lack of increased caspase 3 cleavage and TUNEL labeling (data not shown).

Because no increase in apoptosis was detected and cell cycle proteins Cdc2 and Cyclin B1 were decreased by APE1/Ref-1 inhibition, cell cycle analysis was performed using Propidium Iodide (PI) staining. PC-3 and C4-2 cells were treated with APX2009 (9 μM and 14 μM, respectively) for 48 hours and then stained with PI and analyzed by flow cytometry (FIG. 5C). It was found that the percentage of cells in G1 significantly increased, p<0.05 via Student's t-test, from 58 to 68% and 63 to 74% in PC3 and C4-2 cells, respectively, indicating G1 arrest of prostate cancer cells in response to APE1/Ref-1 inhibition. These effects on the cell cycle progression are similar to other recent reports of APE1/Ref-1 redox inhibition in cancer.

APX2009 Reduces Survivin mRNA Expression and Perturbs NFκB Activity

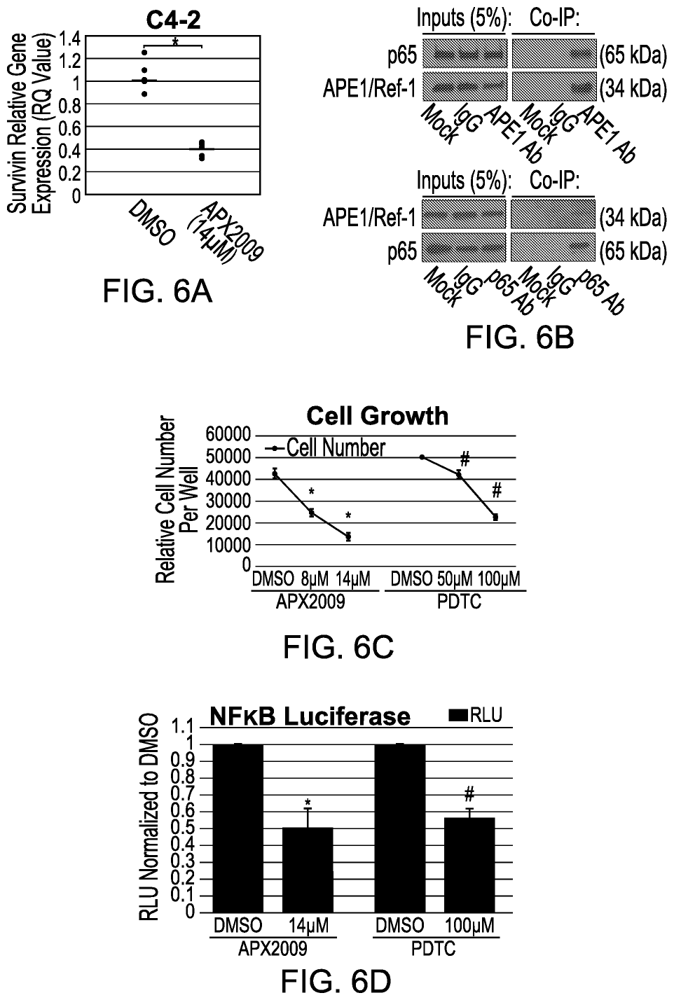

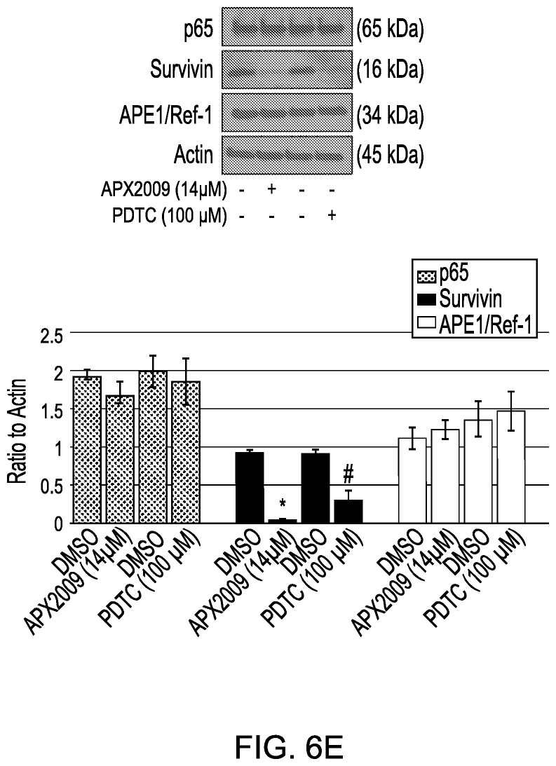

Based on the observation that inhibition of APE1/Ref-1 reduced survivin protein levels, the mechanism by which APE1/Ref-1 regulates survivin expression, and ultimately, cell growth was determined. It was hypothesized that APE1/Ref-1's redox control of transcription factors like NFκB would decrease survivin transcript levels. C4-2 cells were treated with vehicle or APX2009 IC₅₀ (14 μM) for 12 hours. RNA was collected and RT-qPCR was performed using a primer/probe set for survivin (BIRC5) and HPRT1 for the reference gene (FIG. 6A) using the conditions suggested by the SuperScript III Platinum One-Step qRT-PCR System (Invitrogen). Survivin mRNA was significantly reduced upon treatment with the relative quantity (RQ) value of <0.5. Survivin has been shown in other cancers to be regulated by NFκB, and NFκB is regulated by APE1/Ref-1 redox. Therefore, we evaluated the ability of these two proteins to interact physically with each other. In FIG. 6B, it was demonstrated via co-immunoprecipitation that APE1/Ref-1 interacts with NFκB subunit p65 when using an APE1/Ref-1 antibody and in reverse experiments using a p65 antibody.

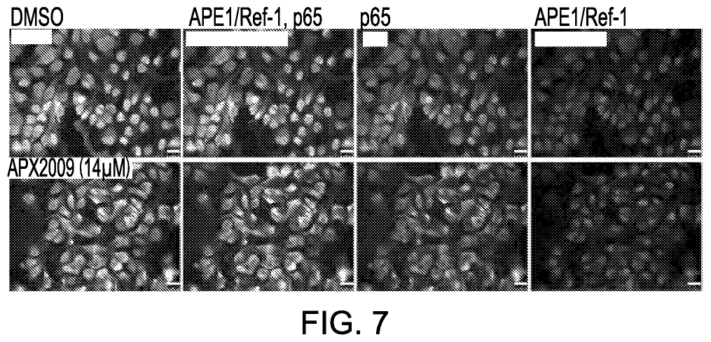

To determine if NFκB signaling is responsible for cell growth and regulated by APE1/Ref-1 redox activity, C4-2 cells were treated with increasing concentrations of APX2009 and NFκB inhibitor ammonium pyrrolidinedithiocarbamate (PDTC) to determine the respective growth inhibition (FIG. 6C). NFκB activity was analyzed in the presence of these two drugs and a significant two-fold decrease in NFκB-driven luciferase activity was found (FIG. 6D). To further confirm a role of NFκB in regulating survivin protein levels, C4-2 cells were treated with 14 μM APX2009 and 100 μM PDTC for 48 hours and a significant 95% and 67% reduction in survivin protein levels, respectively, was observed (FIG. 6E). In addition, the cellular localization of both NFκB and APE1/Ref-1 upon treatment with APX2009 was assessed (FIG. 7). p65 and APE1/Ref-1 were found to be co-localized in the nucleus; however upon treatment with APX2009, p65 nuclear localization was diminished, suggesting altered NFκB protein trafficking.

APE1/Ref-1 Redox Inhibition Decreases Survivin Protein Levels and Cell Proliferation In Vivo

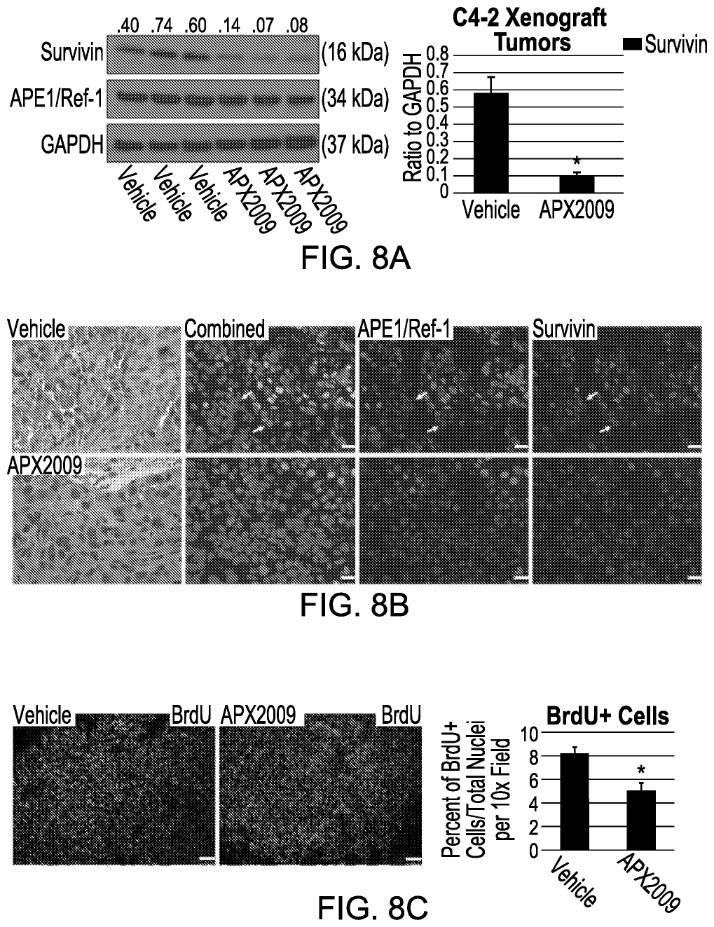

Based on the in vitro data, the role of APE1/Ref-1 redox activity in cell proliferation and survivin protein levels in vivo was analyzed using C4-2 subcutaneous xenografts. The data in FIGS. 8A-8C demonstrates that APE1/Ref-1 redox activity also plays a role in cell proliferation and survivin protein levels in vivo. Animals were treated with either APX2009 (25 mg/kg BID) or vehicle for 5 days and then tumors were harvested. Total survivin protein via immunoblotting was significantly reduced (FIG. 8A) when compared to control tumors. Survivin and APE1/Ref-1 localization via immunofluorescence remained nuclear with survivin co-localizing with the chromatin during mitosis (FIG. 8B). Furthermore, BrdU incorporation was significantly reduced from 8.2% to 5.1% in the treatment group demonstrating that inhibition of APE1/Ref-1 redox activity reduces tumor cell proliferation (FIG. 8C)

The above results indicate that APE1/Ref-1 and survivin are overexpressed in primary and metastatic tumors. APE1/Ref-1 was found to be primarily nuclear localized, but cytoplasmic staining was present in the tumors. Currently, the cellular localization of APE1/Ref-1 has not been fully characterized and more research is needed to determine what differential staining patterns mean to the severity of the disease.

Small molecule inhibitors, APX3330 and APX2009, of APE1/Ref-1 redox activity lead to decreased cell proliferation in a concentration-dependent manner and induced G1 cell cycle arrest. APE1/Ref-1 knockdown also inhibited cell proliferation and replicated what was shown with the inhibitors (FIGS. 7A-7C). Furthermore, treatment with APX3330 and APX2009 resulted in the decrease of survival proteins Bcl-2, Mcl-1 and survivin, where survivin was the most consistent among the cell lines (FIGS. 8A & 8B).

Prostatic tumor xenografts treated with APX3330 displayed decreased survivin protein levels via immunoblot and cell proliferation via BrdU staining. APX3330 was used for the in vivo experiments due to its more characterized pharmacokinetic and pharmacodynamics properties. In the future, APX2009 will be used a single agent and in combination with other therapeutics in vivo to validate its in vitro results. Together, these data demonstrates that APE1/Ref-1 redox inhibition in vivo is a viable option to decrease survivin protein levels and ultimately slow down prostatic tumor progression.

In summary, the present disclosure has identified a new role of APE1/Ref-1's redox function in regulating survivin protein levels in human prostate cancer cell lines. Survivin plays an important role in prostate cancer survival and progression. Thus, inhibition of APE1/Ref-1's redox function in combination with the current therapeutics may prove to be a novel treatment strategy in advanced prostate cancer.

1. A method of treating metastatic prostate cancer in a subject in need thereof, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

2. The method as set forth in claim 1, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), [(2E)-2-[(3-methoxy-1,4-dioxo-1,4-dihydronaphthalen-2-yl)methylidene]-N,N-diethylpentanamide] (APX2009), pharmaceutically acceptable salts and pharmaceutically acceptable solvates thereof, and combinations thereof.

3. The method as set forth in claim 1, wherein the APE1/Ref-1 inhibitor is APX3330 and the subject is administered from about 5 μM to about 100 μM APX3330.

4. The method as set forth in claim 1, wherein the APE1/Ref-1 inhibitor is APX2009 and the subject is administered from about 1 μM to about 30 μM APX2009.

5. The method as set forth in claim 1 further comprising administering at least one additional therapeutic agent to the subject.

6. The method as set forth in claim 5, wherein the additional therapeutic agent is a chemotherapeutic agent selected from the group consisting of cyclophosphamide, dexamethasone, vincristine, doxorubicin, methotrexate, cisplatin, carboplatin, doxetaxel, carazitaxel, taxotere, steroids, antiandrogens, anti-LHRH, ionizing radiation, radiation drugs, and combinations thereof.

7. (canceled)

8. A method of decreasing cancer cell proliferation in a subject in need thereof, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

9. The method as set forth in claim 8, wherein the cancer cell is a metastatic prostate cancer cell.

10. The method as set forth in claim 8, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), [(2E)-2-[(3-methoxy-1,4-dioxo-1,4-dihydronaphthalen-2-yl)methylidene]-N,N-diethylpentanamide] (APX2009), pharmaceutically acceptable salts and pharmaceutically acceptable solvates thereof, and combinations thereof.

11. The method as set forth in claim 8, wherein the APE1/Ref-1 inhibitor is APX3330 and the subject is administered from about 5 μM to about 100 μM APX3330.

12. The method as set forth in claim 8, wherein the APE1/Ref-1 inhibitor is APX2009 and the subject is administered from about 1 μM to about 30 μM APX2009.

13. The method as set forth in claim 8 further comprising administering at least one additional therapeutic agent to the subject.

14. The method as set forth in claim 13, wherein the additional therapeutic agent is a chemotherapeutic agent selected from the group consisting of cyclophosphamide, dexamethasone, vincristine, doxorubicin, methotrexate, cisplatin, carboplatin, doxetaxel, carazitaxel, taxotere, steroids, antiandrogens, anti-LHRH, ionizing radiation, radiation drugs, and combinations thereof.

15. (canceled)

16. A method of reducing survivin expression in a subject in need thereof, the method comprising administering to the subject an effective amount of an apurinic/apyrimidinic endonuclease 1 redox factor 1 (APE1/Ref-1) inhibitor, pharmaceutically acceptable salts or pharmaceutically acceptable solvates thereof, which selectively inhibits the redox function of Ape1/Ref-1.

17. The method as set forth in claim 16, wherein the subject has metastatic prostate cancer.

18. The method as set forth in claim 16, wherein the APE1/Ref-1 inhibitor is selected from the group consisting of 3-[(5-(2,3-dimethoxy-6-methyl1,4-benzoquinoyl)]-2-nonyl-2-proprionic acid (APX3330), [(2E)-2-[(3-methoxy-1,4-dioxo-1,4-dihydronaphthalen-2-yl)methylidene]-N,N-diethylpentanamide] (APX2009), pharmaceutically acceptable salts and pharmaceutically acceptable solvates thereof, and combinations thereof.

19. The method as set forth in claim 16, wherein the APE1/Ref-1 inhibitor is APX3330 and the subject is administered from about 5 μM to about 100 μM APX3330.

20. The method as set forth in claim 16, wherein the APE1/Ref-1 inhibitor is APX2009 and the subject is administered from about 1 μM to about 30 μM APX2009.

21. The method as set forth in claim 16 further comprising administering at least one additional therapeutic agent to the subject.

22. The method as set forth in claim 21, wherein the additional therapeutic agent is a chemotherapeutic agent selected from the group consisting of cyclophosphamide, dexamethasone, vincristine, doxorubicin, methotrexate, cisplatin, carboplatin, doxetaxel, carazitaxel, taxotere, steroids, antiandrogens, anti-LHRH, ionizing radiation, radiation drugs, and combinations thereof.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)
